(19) United States
(12) Patent Application Publication
(10) Pub. No.: US 2025/0195878 A1
(43) Pub. Date: Jun. 19, 2025

(54) WEARABLE ELECTRICAL STIMULATION DEVICE AND METHOD

(71) Applicants: Arizona Board of Regents on behalf of Arizona State University, Scottsdale, AZ (US); CommonSpirit Health, CHICAGO, IL (US)

(72) Inventors: Atharva Ajay Wani, Tempe, AZ (US); Niteesh Nigam, Tempe, AZ (US); Onkar Chavan, Tempe, AZ (US); Yash Rahul Bellap, Tempe, AZ (US); Joelly Lobato de Faria, Chandler, AZ (US); Ishan Swanand Vyas, Tempe, AZ (US); Suhrud Parag Joglekar, Tempe, AZ (US)

(21) Appl. No.: 18/987,506

(22) Filed: Dec. 19, 2024

Related U.S. Application Data
(60) Provisional application No. 63/611,833, filed on Dec. 19, 2023.

(51) Int. Cl. A61N 1/04

(52) U.S. Cl. CPC A61N 1/0484; A61N 1/0452

(57) ABSTRACT

A wearable device includes a flexible garment configured to fit around at least a portion of a subject's hand, a first stimulation pad having an electrode and attached to a first location of the flexible garment, and a second stimulation pad having an electrode and attached to a second location of the flexible garment wherein, when the flexible garment is positioned on the subject's hand, the first location of the flexible garment is adjacent the thenar eminence of the subject's hand, and wherein the first stimulation pad is configured to deliver electrical stimulus to muscle tissue of the thenar eminence, and wherein, when the flexible garment is positioned on the subject's hand, the second location of the flexible garment is adjacent the hypothenar eminence of the subject's hand, and wherein the second stimulation pad is configured to deliver electrical stimulus to muscle tissue of the hypothenar eminence.

### CROSS-REFERENCE TO RELATED APPLICATIONS

This application claims priority to U.S. Provisional Application No. 63/611,833 filed on Dec. 19, 2023, incorporated herein by reference in its entirety.

### BACKGROUND OF THE DISCLOSURE

There is a growing number of people experiencing hand weakness due to stoke or spinal cord injuries. Present devices that address hand weakness recovery via electrical stimulation (e-stim) are bulky, expensive, non-portable, non-ergonomic, and generally unsuitable for in home use by a patient. Furthermore, present devices utilize sticky and wet e-stim pads that need to be stuck to a patient's skin and changed frequently and cause discomfort to the user. Thus, there is a need in the art for improved electrical stimulation devices for in home use.

### SUMMARY OF THE DISCLOSURE

Some embodiments of the disclosure disclosed herein are set forth below, and any combination of these embodiments (or portions thereof) may be made to define another embodiment.

A wearable device includes a flexible garment configured to fit around at least a portion of a subject's hand, a first stimulation pad having an electrode and attached to a first location of the flexible garment, and a second stimulation pad having an electrode and attached to a second location of the flexible garment wherein, when the flexible garment is positioned on the subject's hand, the first location of the flexible garment is adjacent the thenar eminence of the subject's hand, and wherein the first stimulation pad is configured to deliver electrical stimulus to muscle tissue of the thenar eminence, and wherein, when the flexible garment is positioned on the subject's hand, the second location of the flexible garment is adjacent the hypothenar eminence of the subject's hand, and wherein the second stimulation pad is configured to deliver electrical stimulus to muscle tissue of the hypothenar eminence.

In some embodiments, the device further includes a tightening unit, the tightening unit comprising a control unit and a plurality of tightening lines, wherein the plurality of tightening lines are connected to the control unit and the flexible garment, and wherein the control unit is configured to adjust the tension on the plurality of tightening lines. In some embodiments, the device further includes one or more pressure sensors attached to the flexible garment and configured to measure pressure on the skin of the subject, wherein the one or more sensors are connected to the control unit. In some embodiments, the control unit is voice-activated for adjusting the tension on the plurality of tightening lines and activating the electrodes of the stimulation pads. In some embodiments, the control unit adjusts the tension on the plurality of tightening lines based on one more pressure signals received from the one or more pressure sensors.

In some embodiments, the tightening unit comprises a housing having a plurality of holes, wherein the plurality of tightening lines extend through the plurality of holes to connect to the control unit positioned inside the housing. In some embodiments, the control unit comprises at least one motor connected to the plurality of tightening lines, wherein the at least one motor is configured to rotate to adjust the tension on the plurality of tightening lines. In some embodiments, the tightening unit and the plurality of tightening lines are removably connected to the flexible garment.

In some embodiments, the first stimulation pad comprises a body having one or more curved surfaces configured to nest on a portion of the subject's hand. In some embodiments, the second stimulation pad comprises a body having one or more curved surfaces configured to surround a portion of the subject's hand. In some embodiments, the flexible garment comprises a cut-out region for the thumb of the subject's hand.

In some embodiments, the device further includes a third stimulation pad having an electrode and attached to a third location of the flexible garment, wherein a portion of the flexible garment covers a region of the subject's arm, and the third location of the flexible garment is adjacent the forearm of the subject's arm, and wherein the third stimulation pad is configured to deliver electrical stimulus to muscle tissue of the arm. In some embodiments, the device further includes a fourth stimulation pad having an electrode and attached to a fourth location of the flexible garment, wherein the fourth location of the flexible garment is adjacent the forearm of the subject's arm, and wherein the fourth stimulation pad is configured to deliver electrical stimulus to muscle tissue of the arm. In some embodiments, the third location of the flexible garment is adjacent the medial forearm of the subject's arm, and the fourth location of the flexible garment is adjacent the lateral forearm of the subject's arm.

In some embodiments, the electrodes of the electrical stimulation pads positioned in the first and third locations of the flexible garment deliver electrical stimulus in a pair, and the electrodes in the second and fourth locations of the flexible garment deliver electrical stimulus in a pair. In some embodiments, the third and fourth stimulation pads each comprise a body having a convex surface. In some embodiments, the first location and second location on the flexible garment are separated by a distance ranging between 1 cm and 10 cm. In some embodiments, the first location and third location on the flexible garment are separated by a distance ranging between 4 cm and 50 cm, and the third location and fourth location on the flexible garment are separated by a distance ranging between 1 cm and 15 cm.

In some embodiments, the electrodes of the stimulation pads are configured to deliver an electrical stimulus in the range 0-100 mA at 0-50 V. In some embodiments, the electrodes of the stimulation pads are configured to deliver an electrical stimulus at a 40 Hz frequency with 250 μS pulse width.

### BRIEF DESCRIPTION OF THE DRAWINGS

The foregoing purposes and features, as well as other purposes and features, will become apparent with reference to the description and accompanying figures below, which are included to provide an understanding of the disclosure and constitute a part of the specification, in which like numerals represent like elements, and in which:

FIG. 1 depicts an exemplary electrical stimulation device in accordance with some embodiments.

FIG. 2 depicts an exemplary pad connection mechanism in accordance with some embodiments.

FIG. 3 depicts an exemplary control unit in accordance with some embodiments.

FIGS. 4A-4E depict back (FIG. 4A), bottom (FIG. 4B), front (FIG. 4C), side (FIG. 4D), and top (FIG. 4E) views of an exemplary little finger electrical stimulation pad in accordance with some embodiments.

FIGS. 5A-5F depict back (FIG. 5A), bottom (FIG. 5B), front (FIG. 5C), first side (FIG. 5D), second side (FIG. 5E), and top (FIG. 5F) views of an exemplary thumb electrical stimulation pad in accordance with some embodiments.

FIG. 6 depict various exemplary electrical stimulation pad designs in accordance with some embodiments.

FIGS. 7A-7E depict perspective (FIG. 7A), top (FIG. 7B), front (FIG. 7C), bottom (FIG. 7D), and bottom perspective (FIG. 7E) views of an exemplary top portion for an electrical stimulation pad in accordance with some embodiments.

FIGS. 8A-8E depict perspective (FIG. 8A), top (FIG. 8B), first side (FIG. 8C), second side (FIG. 8D), and cutaway (FIG. 8E) views of an exemplary bottom portion for an electrical stimulation pad in accordance with some embodiments.

FIGS. 9A-9E depict perspective (FIG. 9A), top (FIG. 9B), side (FIG. 9C), bottom (FIG. 9D), and bottom perspective (FIG. 9E) views of an exemplary top portion for an electrical stimulation pad in accordance with some embodiments.

FIGS. 10A-10D depict top (FIG. 10A), side (FIG. 10B), bottom (FIG. 10C), and cutaway (FIG. 10D) views of an exemplary bottom portion for an electrical stimulation pad in accordance with some embodiments.

FIGS. 11A-11B depict perspective (FIG. 11A), and side (FIG. 11B) views of an exemplary electrical stimulation pad in accordance with some embodiments.

FIGS. 12A and 12B depict exemplary pad placement locations in accordance with some embodiments.

FIG. 13 depicts an exemplary computing environment in which aspects of the disclosure may be practiced.

### DETAILED DESCRIPTION OF THE DISCLOSURE

It is to be understood that the figures and descriptions of the present disclosure have been simplified to illustrate elements that are relevant for a clearer comprehension of the present disclosure, while eliminating, for the purpose of clarity, many other elements found in devices, systems and methods of electrical stimulation for patients with hand weakness due to stroke or spinal cord injury. Those of ordinary skill in the art may recognize that other elements and/or steps are desirable and/or required in implementing the present disclosure. However, because such elements and steps are well known in the art, and because they do not facilitate a better understanding of the present disclosure, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20%, +10%, +5%, +1%, and +0.1% from the specified value, as such variations are appropriate.

Ranges: throughout this disclosure, various aspects of the disclosure can be presented in a range format. It should be understood that the description in range format is merely for convenience and brevity and should not be construed as an inflexible limitation on the scope of the disclosure. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The terms “patient,” “subject,” “individual,” and the like are used interchangeably herein, and refer to any animal amenable to the systems, devices, and methods described herein. The patient, subject or individual may be a mammal, and in some instances, the mammal may be a human.

As described herein, when a general finger is referenced (i.e. thumb, pointer, little . . . ) related to an electrical stimulation pad (in some example referred to as an “e-stim” pad) or pad placement location, the general finger referenced includes the specific finger, and any suitable tendons, ligaments, musculature, nerves or similar which actuates movement of said finger. For example, when a thumb electrical stimulation pad is described, this includes an electrical stimulation pad on a thumb side of the device, proximate to the thumb, and any biological actuation mechanisms (musculature, ligaments, . . . ) associated with the thumb.

Some aspects of the present disclosure may be made using an additive manufacturing (AM) process. Among the most common forms of additive manufacturing are the various techniques that fall under the umbrella of “3D Printing”, including but not limited to stereolithography (SLA), digital light processing (DLP), fused deposition modelling (FDM), selective laser sintering (SLS), selective laser melting (SLM), electronic beam melting (EBM), and laminated object manufacturing (LOM). These methods variously “build” a three-dimensional physical model of a part, one layer at a time, providing significant efficiencies in rapid prototyping and small-batch manufacturing. AM also makes possible the manufacture of parts with features that conventional subtractive manufacturing techniques (for example CNC milling) are unable to create.

Suitable materials for use in AM processes include, but are not limited to, using materials including but not limited to nylon, polyethylene terephthalate (PET), acrylonitrile butadiene styrene (ABS), resin, polylactic acid (PLA), polystyrene, and the like. In some embodiments, an AM process may comprise building a three-dimensional physical model from a single material, while in other embodiments, a single AM process may be configured to build the three-dimensional physical model from more than one material at the same time.

The present disclosure serves to address numerous complaints by healthcare professionals and subjects (e.g., patients) relating to systems and methods for electrical stimulation of subjects. The primary complaint from the subjects was about the sticky, wet electrical stimulation pads that needed to be stuck to the skin and changed frequently. The primary complaint from the healthcare professional's perspective was the bulky, expensive, and non-portable design of the devices they use and the scarcity of well-integrated home-use devices for subjects. The present disclosure provides designs for electrical stimulation pads and an accessible, ergonomic, and easy to use device.

In some embodiments, the disclosed device is a medical device that is configured to assist patients with chronic weakness of hand due to stroke or spinal cord injury. In some embodiments, the device includes ergonomic electrical stimulation pads that do not need to be changed and do not need to be stuck to the skin. In some embodiments, the device utilizes a wearable garment with voice activation, self-tightening, and/or a sleek design so the patient can wear and use the device without the help of others. For example, in some embodiments, the device includes a wearable garment with a voice-activated self-tightening mechanism.

The combination of the ergonomic and reusable pads, voice activation, and the self-tightening mechanism make the disclosed device unique from existing devices. By combining all these features, the resulting device makes the user much more independent and self-sufficient as they can wear and remove the device on their own without the help of anyone. The device aims to bridge the gap between the products available on the market and the need for a more comfortable and “independent” product by consumers. Electrical stimulation pads can be moisture-controlled, requiring them to be wet under operation, or they can be gel, which can be highly uncomfortable for the users. The disclosed device, which in some embodiments comprises electrical stimulation pads along with several additional attachments, solves the problem of pads that need to be changed frequently. The ergonomic design of the disclosed wearable garment ensures natural grip and provides robust control of actuation by implementing voice and touch sensing.

Aspects of the present disclosure relate to an electric stimulation device used to stimulate muscles in the hands of patients with chronic weakness of hand due to stroke or spinal cord injury. In other aspects, the present disclosure provides electrical stimulation pad designs in accordance with some embodiments. Further, the present disclosure provides voice-activated and pressure-controlled self-tightening mechanisms for wearable garments.

Referring now in detail to the drawings, in which like reference numerals indicate like parts or elements throughout the several views, in various embodiments, presented herein are devices, systems and methods of electrical stimulation for patients with hand weakness due to stroke or spinal cord injury.

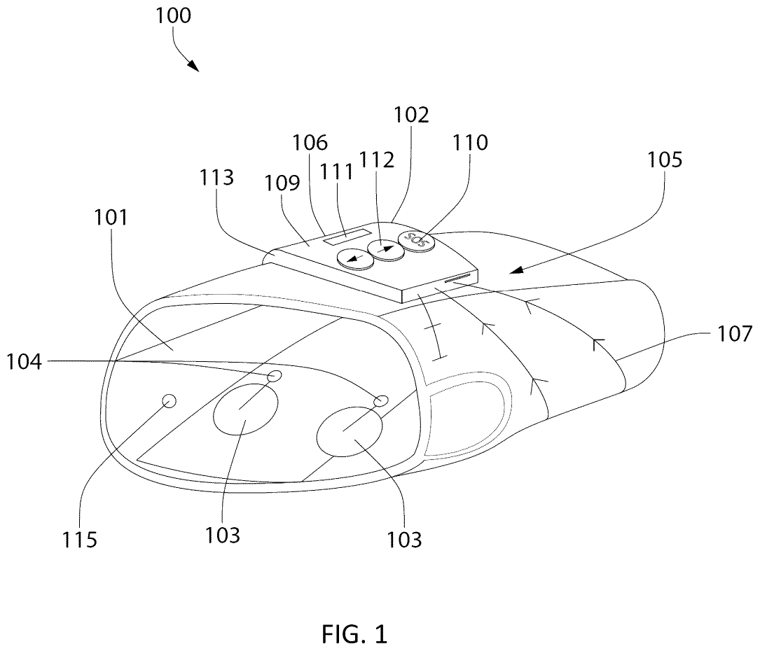

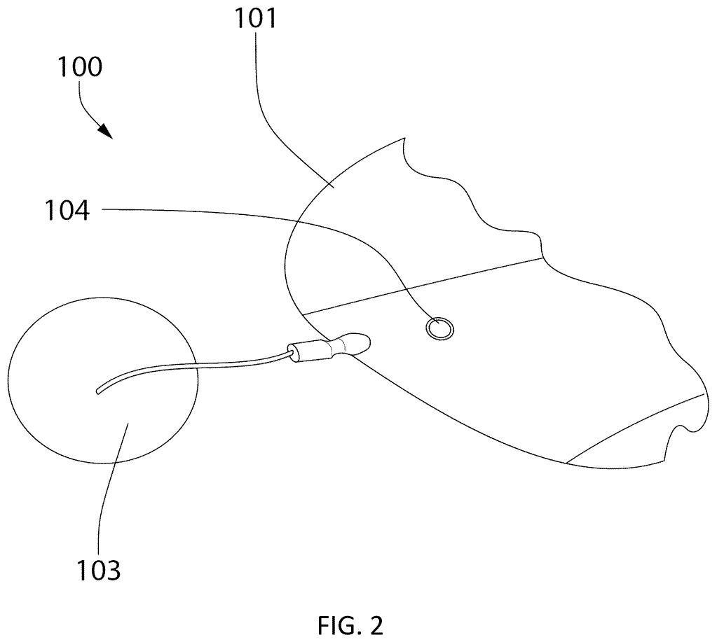

Referring now to FIG. 1, shown is an exemplary electrical stimulation device 100 comprising a wearable garment 101 comprising a flexible structure having an inside and outside surface, and configured to surround at least a portion of a subject (e.g., a hand, a wrist, a forearm of a subject). In some embodiments, device 100 comprises a control unit 102 associated with garment 101, and at least one electrical stimulation pad 103 having an electrode positioned on the inside surface of garment 101 and electrically connected to control unit 102. In some embodiments, each electrical stimulation pad 103 is connected to at least one electrical connector 104, which is electrically connected to control unit 102. In some embodiments, control unit 102 is fixedly and removably positioned on the outside surface of garment 101. Connector 24 may comprise any connector known in the art, including, but not limited to, electrical connector, headphone connector, 2.5 mm headphone connector, or the like. In some embodiments, device 100 comprises one or more pressure sensors 115 attached to garment 101 and connected to control unit 102 configured to measure pressure at the interface of the device and the subject. For example, one or more pressure sensors 115 configured to measure the pressure on the skin of the subject, and/or of the stimulation pad 103 on the skin of the subject. It should be appreciated that the one or more pressure sensors 115 may be positioned anywhere on garment 101.

In some embodiments, device 100 further comprises at least one self-tightening mechanism 105 associated with control unit 102 configured to tighten or loosen garment 101. In some embodiments, self-tightening mechanism 105 comprises a motor 106. In some embodiments, self-tightening mechanism 105 and/or motor 106 is positioned within the housing of control unit 102. In some embodiments, control unit 102 may be referred to as a tightening unit comprising a control unit and/or tightening mechanism 105.

In some embodiments, device 100 further comprises a plurality of tightening lines or strings 107 positioned within or around garment 101. In some embodiments, plurality of strings 107 are connected to tightening mechanism 105 of control unit 102. In some embodiments, the control unit is configured to adjust the tension on the tightening lines. In some embodiments, plurality of strings 107 are connected to motor 106 of tightening mechanism 105. In some embodiments, tightening mechanism 105 is configured to pull the strings in order to tighten garment 101. In some embodiments, motor 106 is configured to rotate to pull the strings in order to tighten garment 101. In some embodiments, control unit 102 and/or motor 106 are voice controlled or activated. In some embodiments, the tightening unit and/or the plurality of tightening lines are removably connected to the garment 101, with a hook and loop structure, hooks, anchors and/or clips.

Aspects of the present disclosure relate to a garment (e.g., a wearable garment) for device 100. It should be appreciated that garment 101 may be sized and/or shaped for fixedly and removably positioning the garment to surround a portion of a subject. For example, the flexible structure of garment 101 may comprise a fabric sleeve or tube comprising a sidewall with inner and outer surfaces, and forming a lumen therethrough wherein a subject's appendage may pass through (e.g., a hand, an arm, a forearm, an elbow, a wrist, a finger, a leg, an ankle, a foot). In some embodiments, garment 101 is sized, shaped, and configured as a glove to be worn on a subject's hand, and comprises a lateral thumb opening, and/or distal finger openings.

In some embodiments, portions of device 100 (e.g., garment 101 and/or electrical stimulation pads 103) comprise an ergonomic design that accommodates for natural curves of the body of the subject. For example, in some embodiments, garment 101 comprises a curved region in the flexible structure, configured to fit snuggly around the hand of the subject. Similarly, other aspects of device 100 may comprise curves or non-flat surfaces to conform to the body of the subject. For example, in some embodiments, electrical stimulation pad 103 is curved to fully make contact with the skin of the subject. In some embodiments, electrical stimulation pad 103 comprises copper plating as the electrode, wherein the electrode is pressed onto the skin of the subject with some pressure to induce current through Neuro Muscular Electric Stimulation (NMES). In some embodiments, electrical stimulation pad 103 comprises elongate strips of copper plating for the electrode, wherein the pad can extend across one or more positions or muscles in the subject. In some embodiments, electric stimulation pad 103 is configured to provide electrical stimulation in the range 0-100 mA at 0-50 V. In some embodiments, electrical stimulation pad 103 is configured to provide electrical stimulation between 0-1000 Hz frequency, with 0-1000 μS pulse width. In some embodiments, electric stimulation pad 103 is configured to provide electrical stimulation at a 40 Hz frequency with 250 μS pulse width. In some embodiments, for safety of the subject, electrical stimulation pad 103 is configured to provide electrical stimulation at 40 Hz frequency with a 250 μS pulse width.

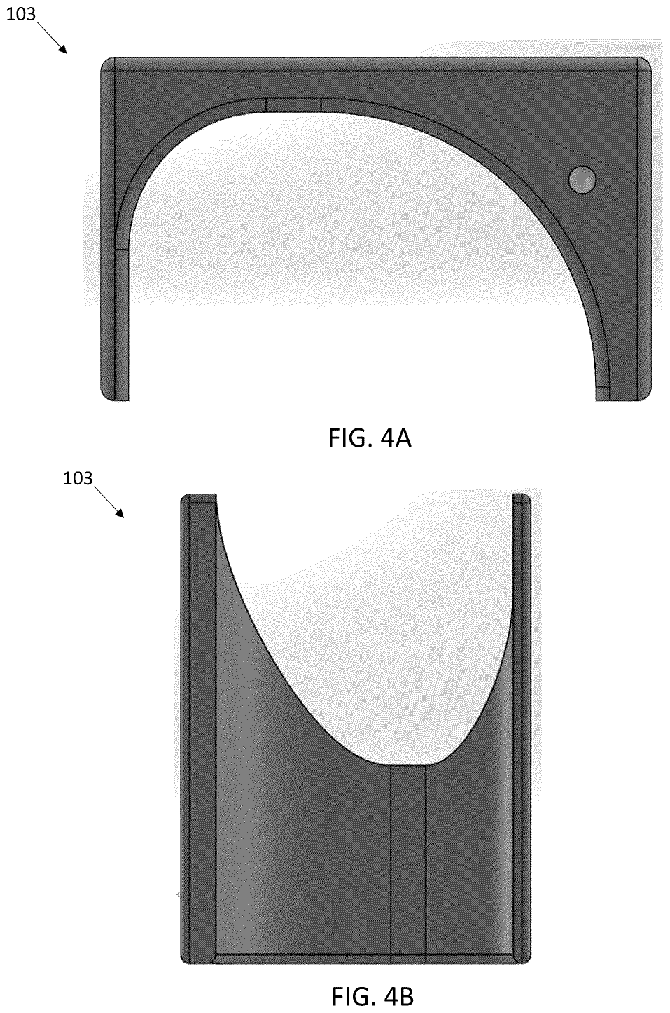

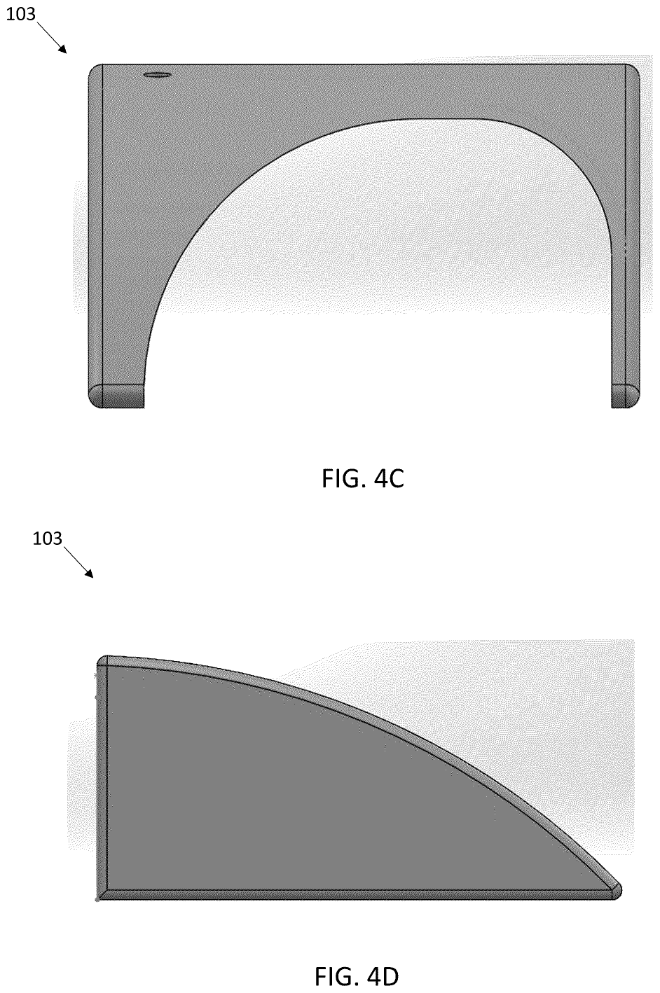

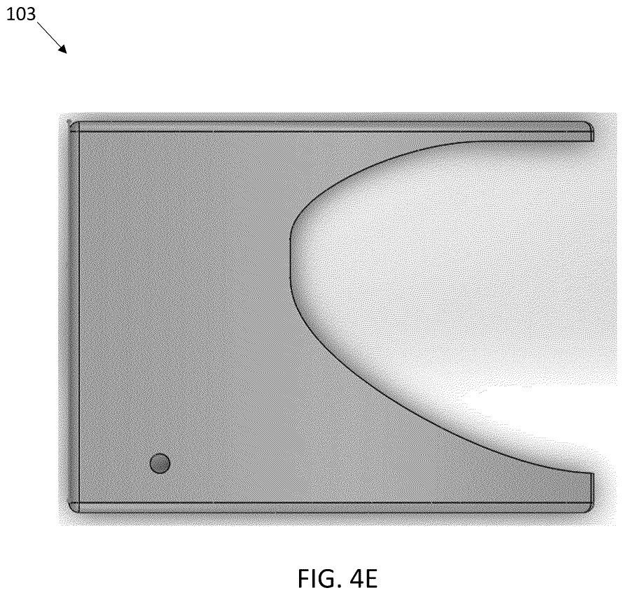

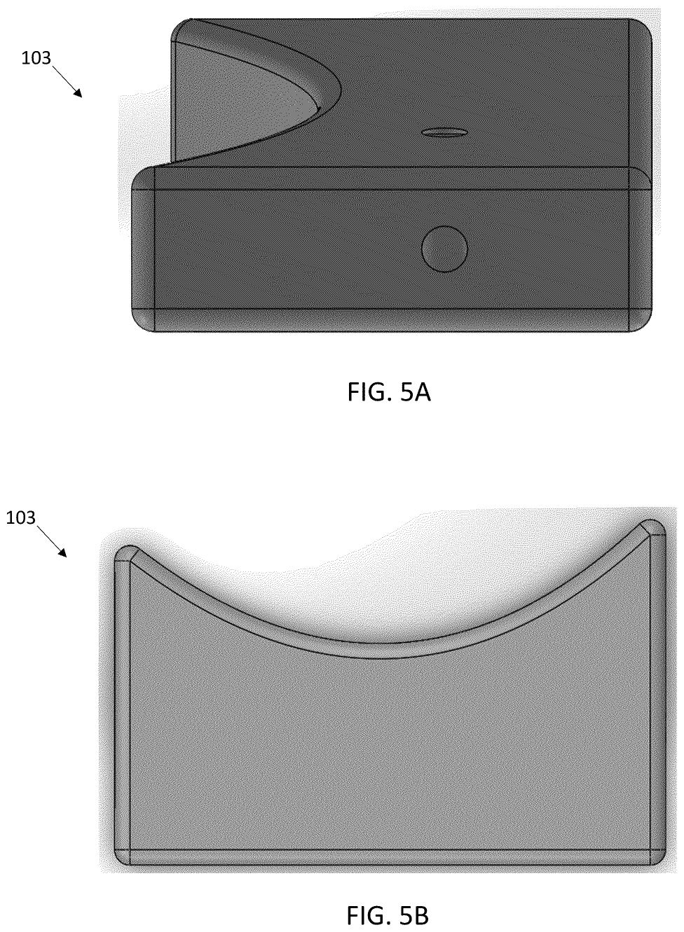

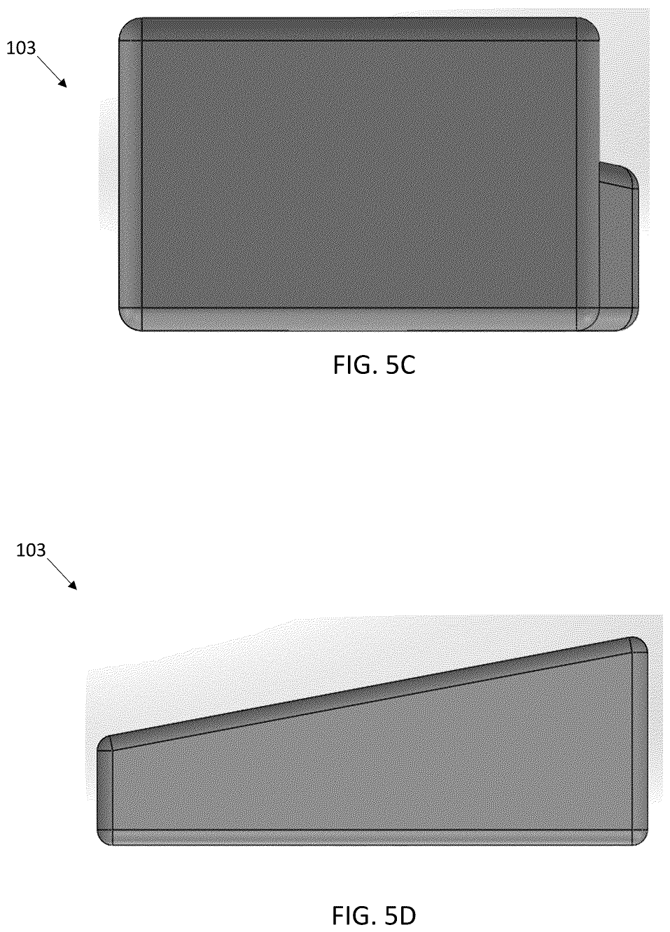

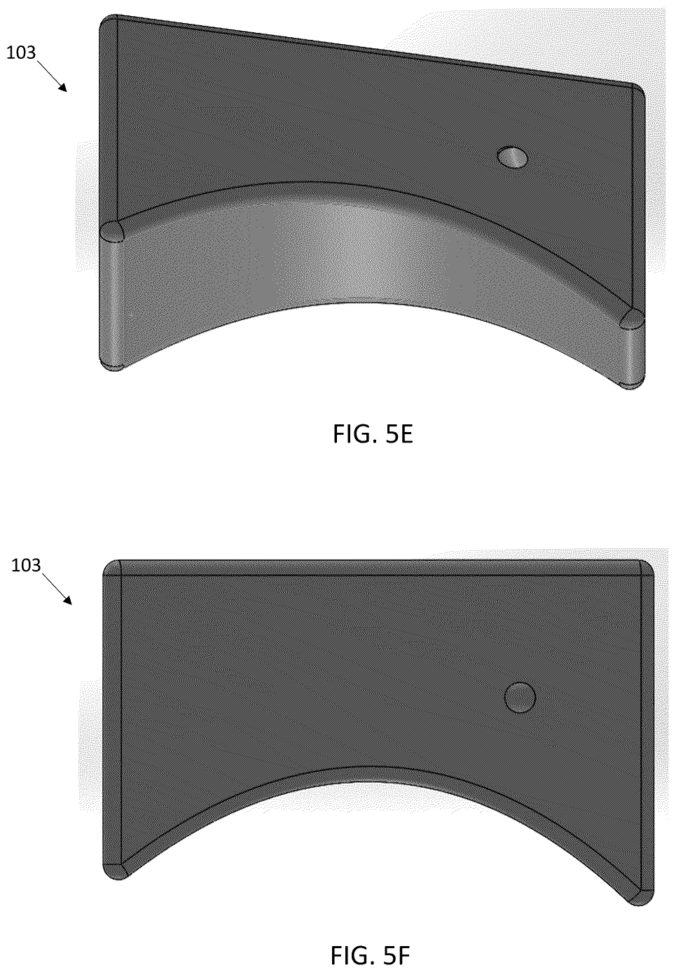

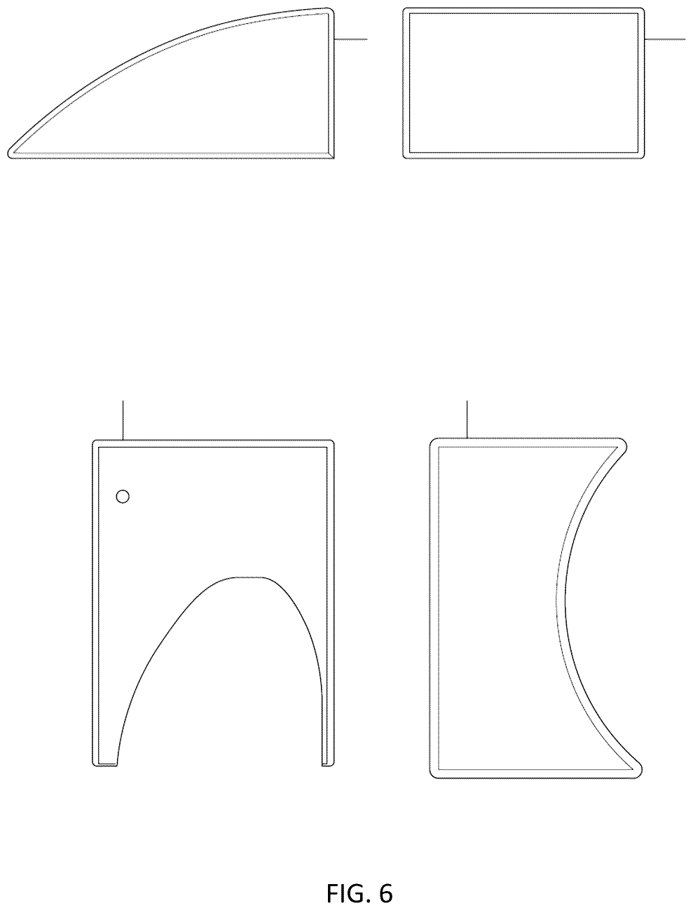

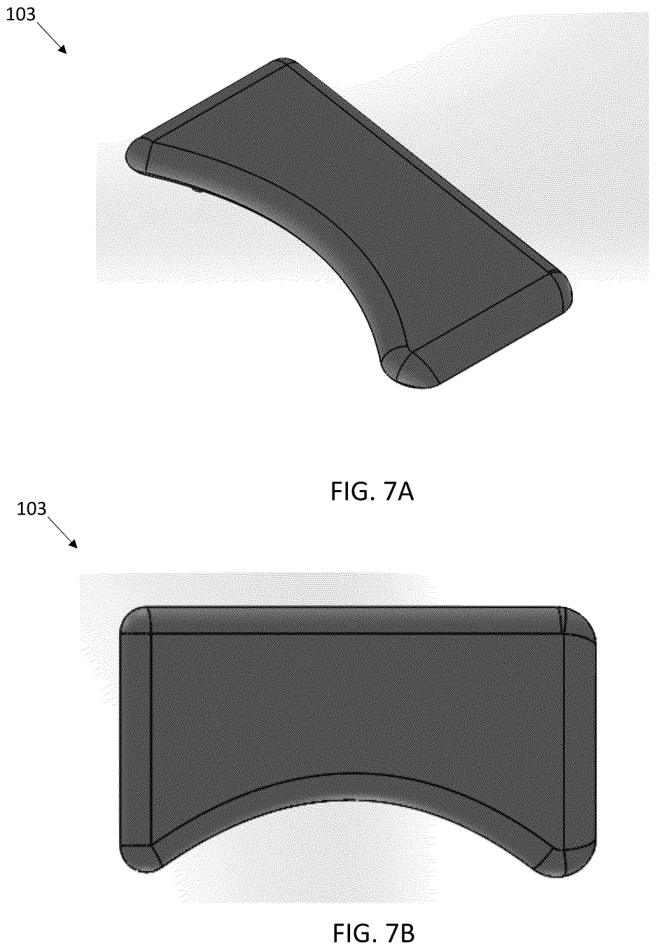

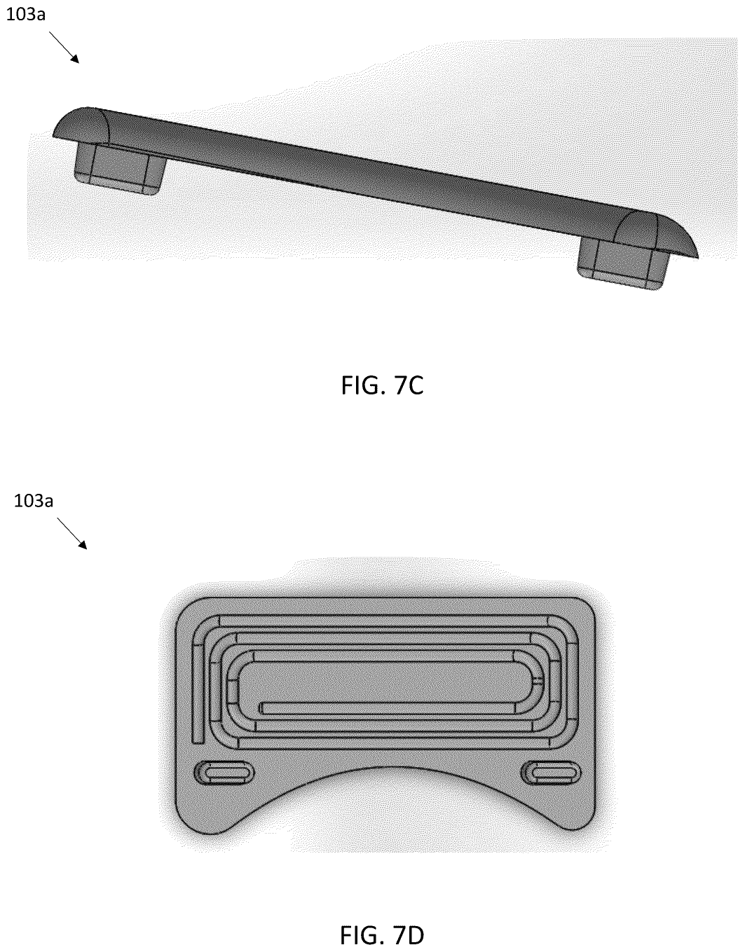

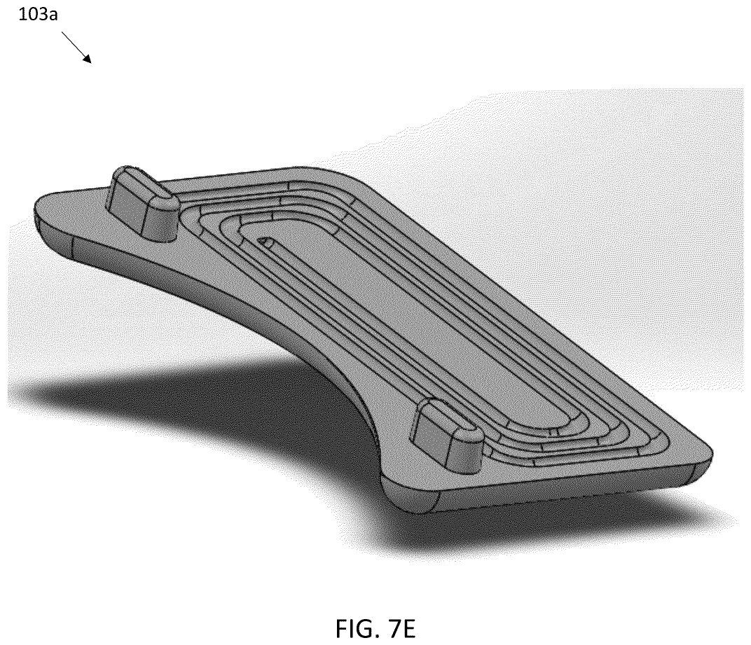

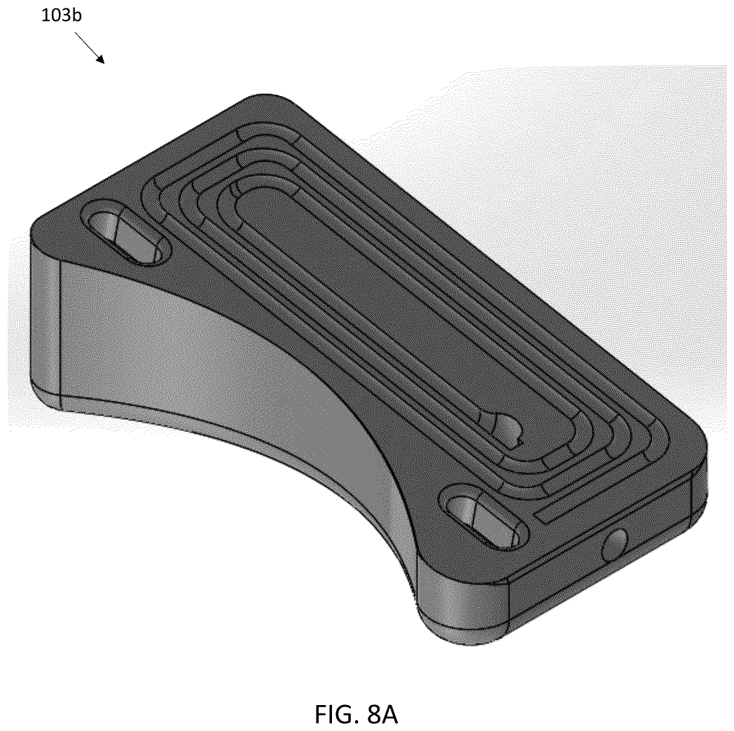

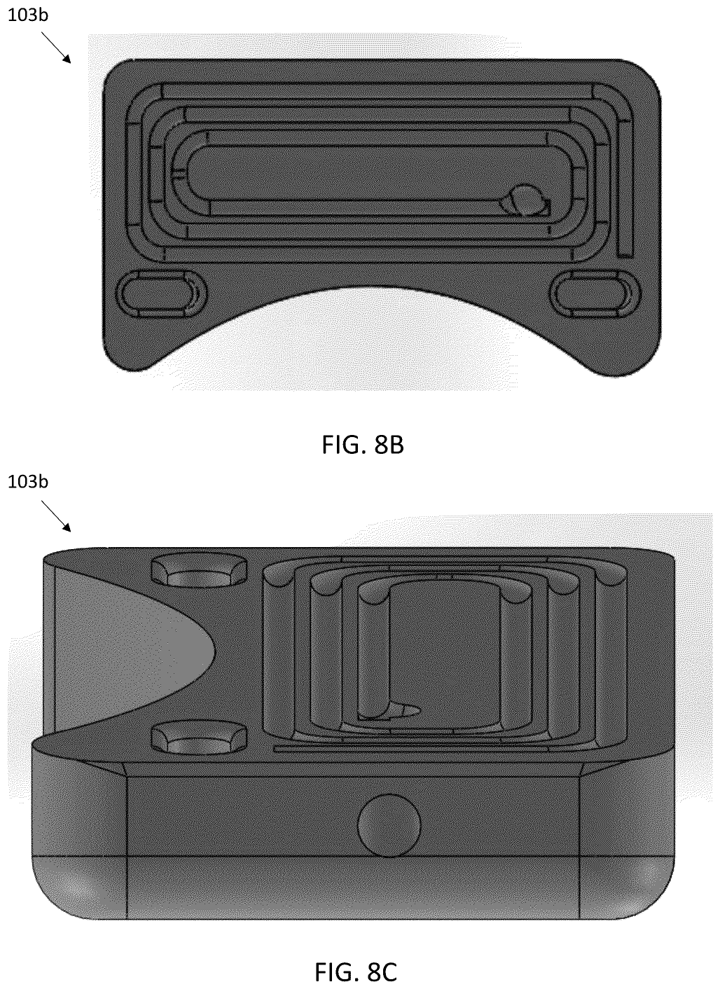

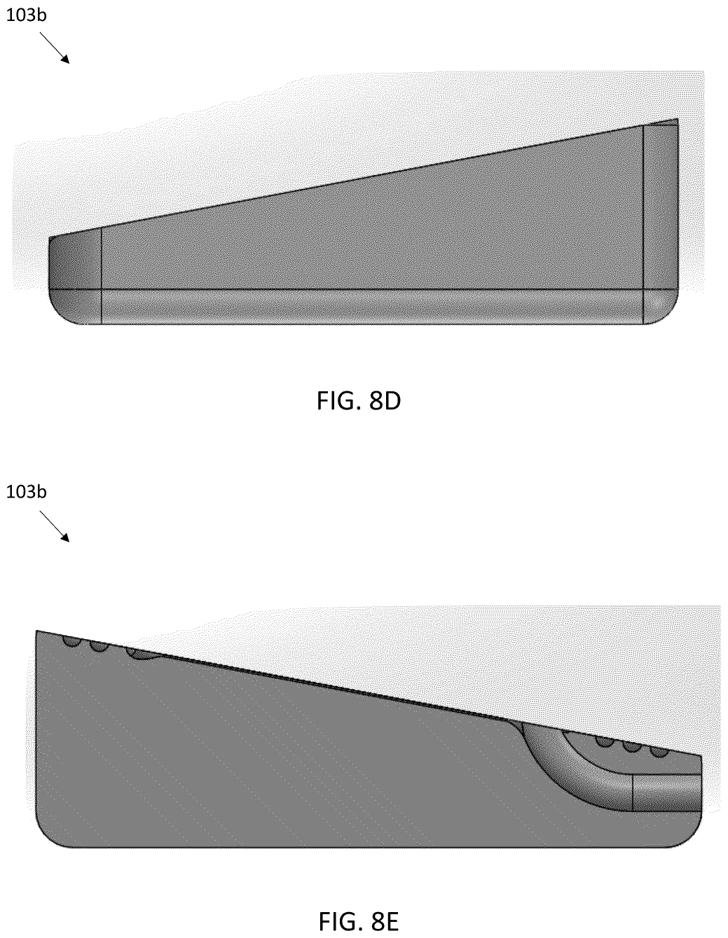

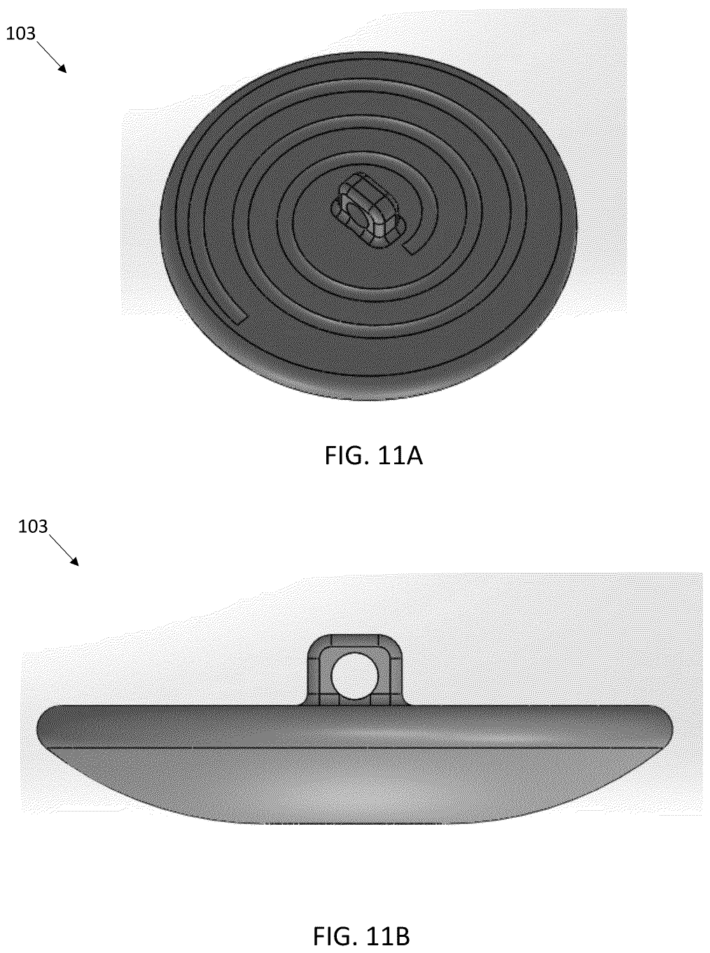

Exemplary pad designs are shown FIGS. 4 through 11. FIGS. 4A-4E depict various views of an exemplary electrical stimulation pad 103 configured for a finger. FIGS. 5A-5F depict various views of an exemplary electrical stimulation pad 103 configured for a thumb. FIG. 6 depict various exemplary electrical stimulation pad designs in accordance with some embodiments. FIGS. 7 & 8 depict an exemplary electrical stimulation pad 103 configured for placement near or on the thenar of a subject. FIGS. 8 & 9 depict an exemplary electrical stimulation pad 103 configured for placement near or on the hypo-thenar of a subject. FIG. 11 depicts an exemplary electrical stimulation pad 103 configured for placement near or on the forearm of a subject.

In some embodiments, each electrical stimulation pad 103 may formed as a single portion or piece, or may be formed as separate portions or pieces that may be fixedly and/or removably coupled or attached. Now referring to FIGS. 7 & 8, depicted is an exemplary electrical stimulation pad 103 configured for placement near or on the thenar of a subject. In some embodiments, electrical stimulation pad 103 comprises a top portion 103a and a bottom portion 103b. FIGS. 7A-7E depict various views of an exemplary top portion 103a, and FIGS. 8A-8E depict various views of an exemplary bottom portion 103b. It should be appreciated that each top portion 103a may be coupled or attached (either fixedly or removably) to a correlated bottom portion 103b via compression or friction fit, or by any mechanical interface known by one of ordinary level of skill in the art. In some embodiments, top portion 103a comprises one or more electrodes, conductive pads or elements, and bottom portion 103b forms a body, housing or a base whereon the pads or elements may reside or be formed upon. In some embodiments, top portion 103a may be composed or manufactured from metal, conductive metal, copper, wire, electrical wire, copper wire, conductive paint, conductive fabric, foam, or the like. In some embodiments, top portion 103a comprises one or more leads, connectors, jacks, pads, stimulation pads, electrical stimulation pads, electrical wires, insulator, channels, holes, conduits, coils, or spiral channels. In some embodiments, bottom portion 103b is composed or manufactured from non-metals, non-conductive metals, composites, plastics, foams, rubbers, or fabrics. In some embodiments, bottom portion 103b comprise any of connectors, jacks, openings, channels, wire conduits, holes, connector holes, spiral channels, or insulator.

In some embodiments, either top portion 103a and/or bottom portion 103b comprise any of slots, tabs, alignment tabs, indexing tabs, posts, holes, grooves, or the like, configured to align the top and bottom portion to couple or attach the portions together. Top portion 103a and/or bottom portion 103b may be formed in any shape, and include curved surfaces and irregular geometries intended for interfacing with a region of interest (ROI) or site on a subject.

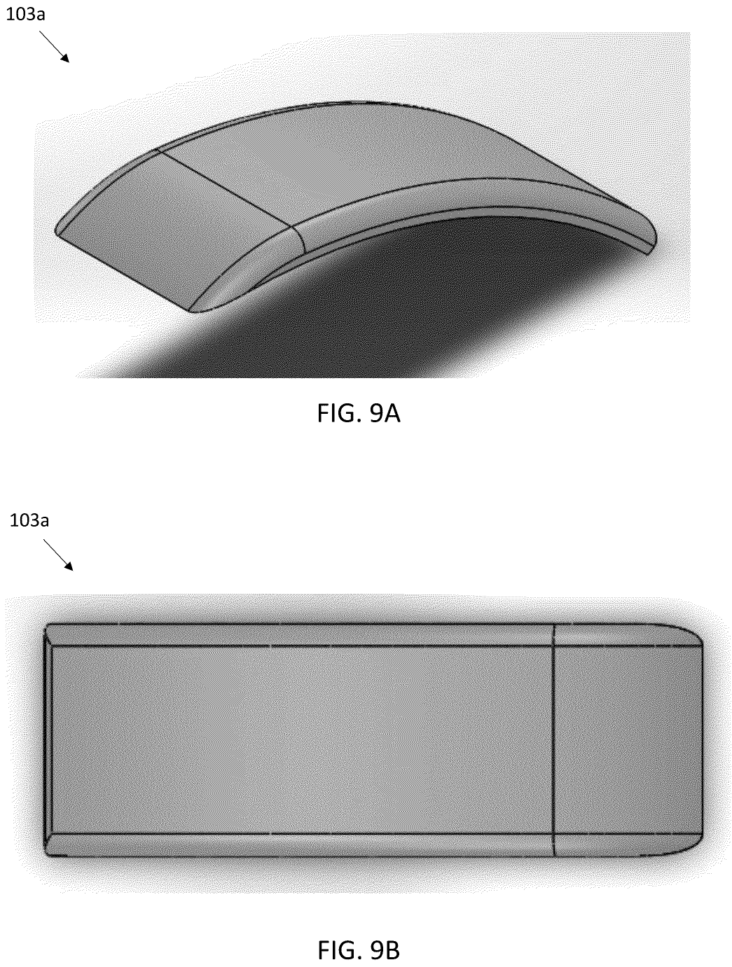

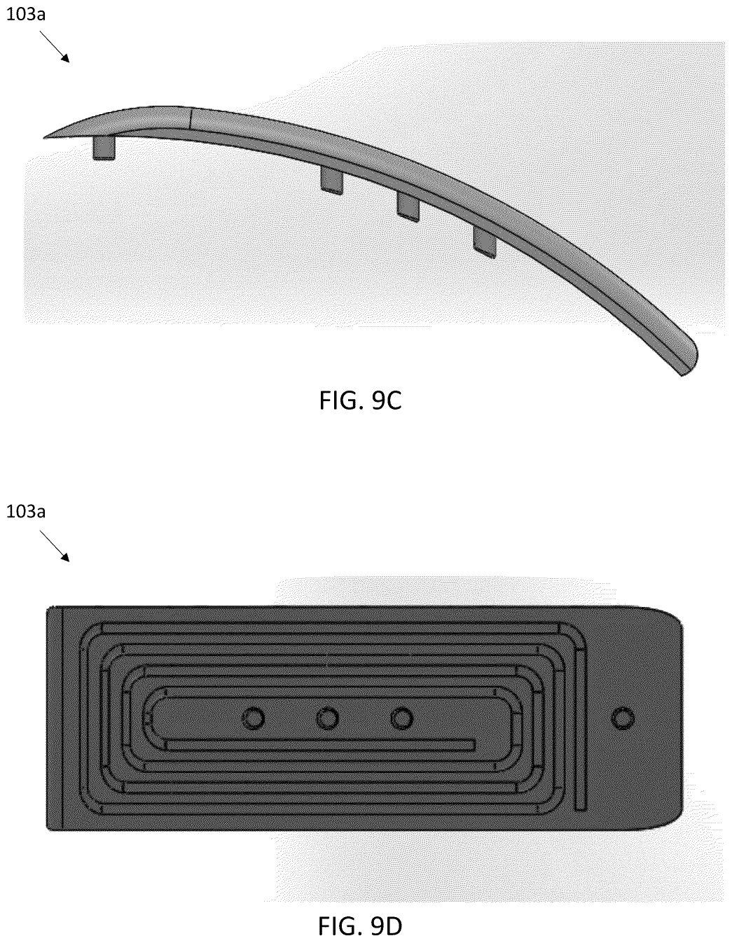

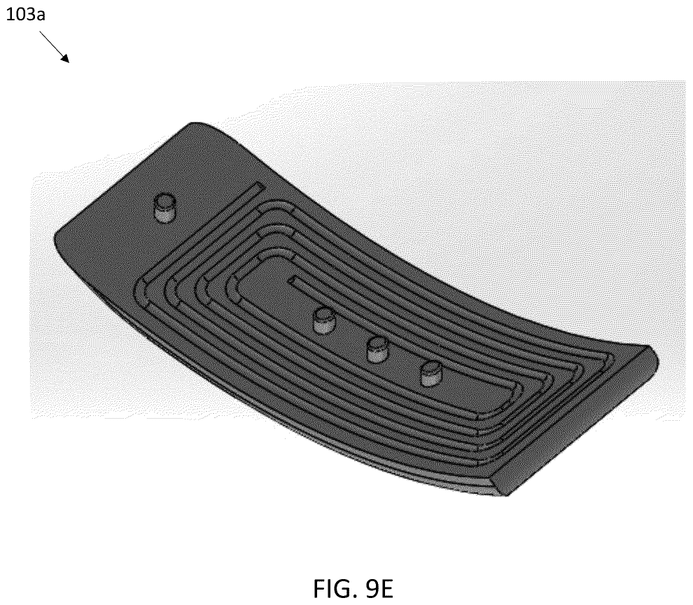

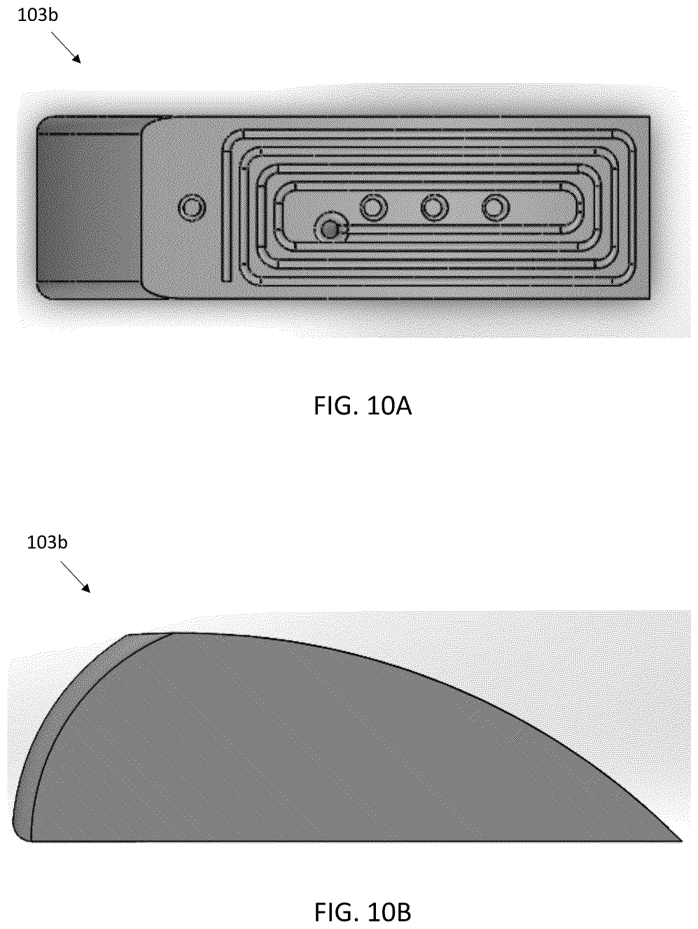

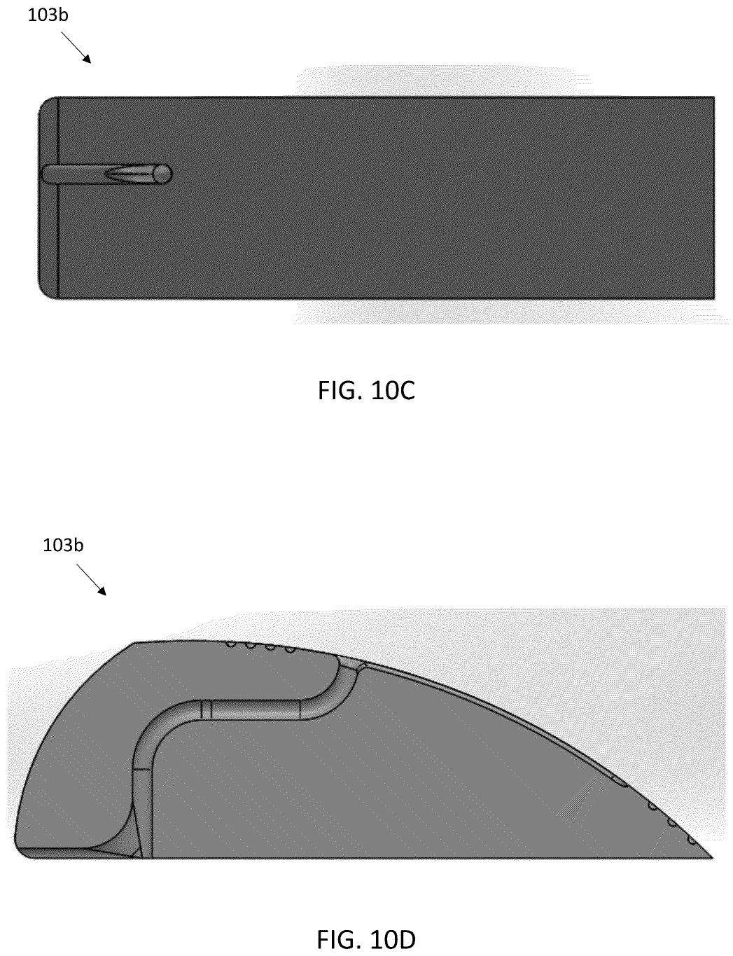

Now referring to FIGS. 8 & 9, depicted is an exemplary electrical stimulation pad 103 configured for placement near or on the hypo-thenar of a subject. FIGS. 9A-9E depict various views of an exemplary top portion 103a, and FIGS. 10A-10D depict an exemplary bottom portion 103b in accordance with some embodiments.

Referring now to FIGS. 11A-11B, shown are various views of an exemplary electrical stimulation pad 103 configured for placement near or on the forearm of the subject in accordance with some embodiments. In some embodiments, electrical stimulation pad 103 may be at least partially formed in the shape of a button and comprises a spiral groove in the button for electrical wire routing.

The one or more electrical stimulation pad 103 may be formed, comprised of, or manufactured from one or more materials selected from plastic, metal, conductive metal, non-conductive metal, foam, rubber, alloy, fabric, PLA, Stainless steel, copper, selective laser sintering (SLS) metal, SS-306, electrical wire, conductive wire, copper wire, conductive paint, conductive fabric, foam, or insulator.

In some embodiments, the placement of the electrical stimulation pads provides exemplary configurations and arrangements for NMES, providing numerous benefits over typical NMES configurations. In some embodiments, the electrical stimulation pads 103 are placed on the Thenar and Hypo-thenar muscles in the hand, in order to induce a grip that is more natural and functional to the subject.

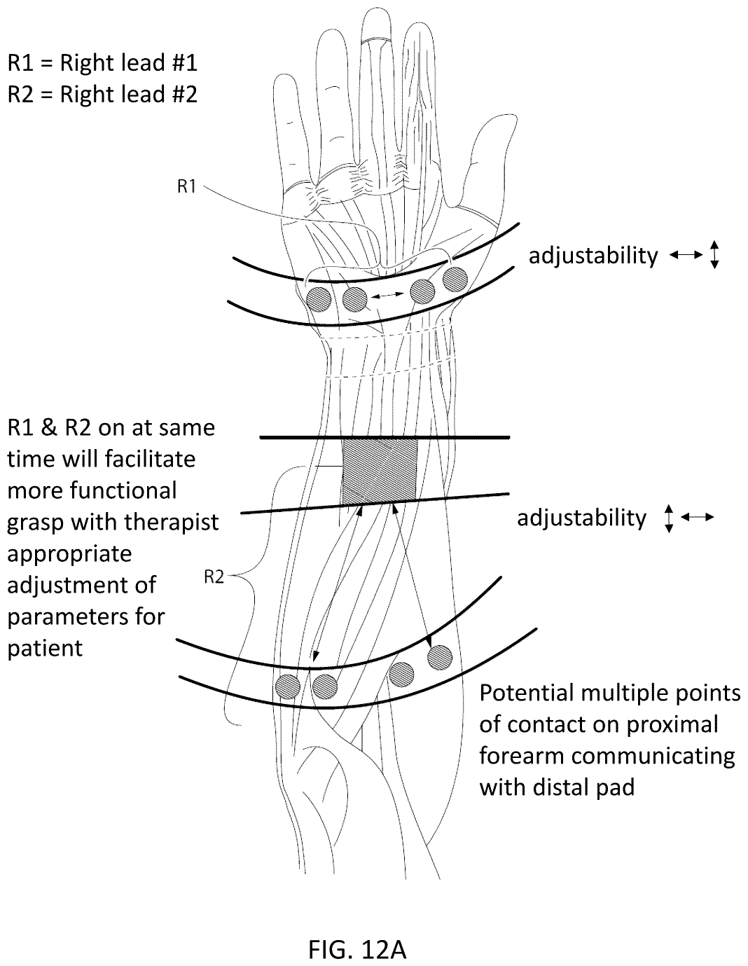

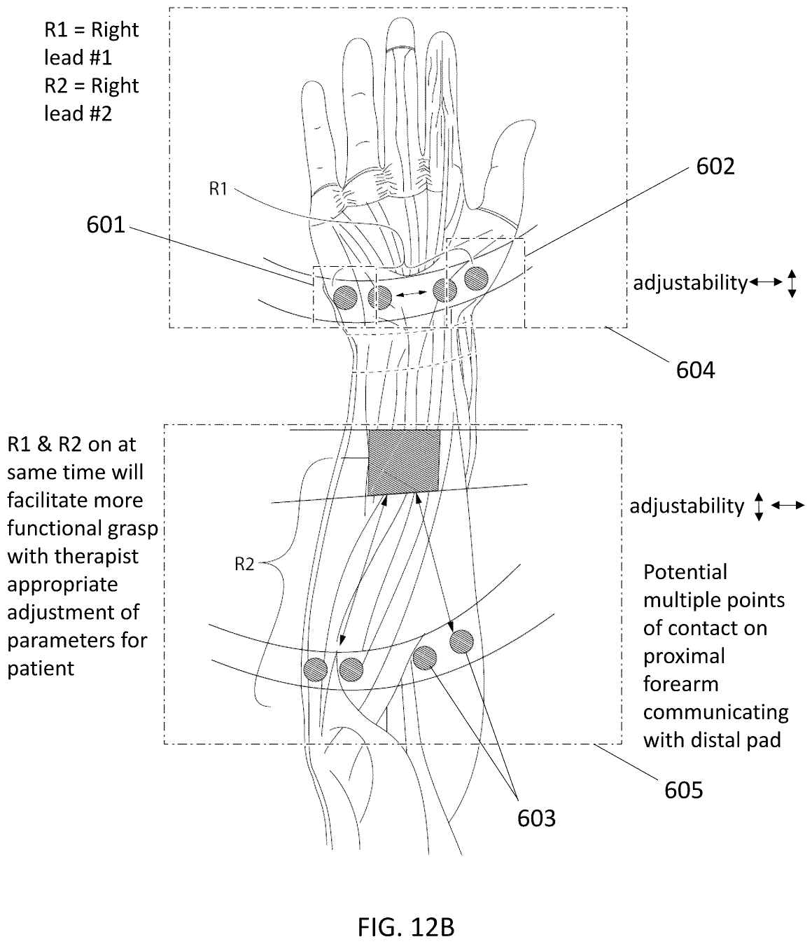

Referring now to FIG. 12B, shown are exemplary pad placement locations in accordance with some embodiments. In FIG. 12B, region 601 is the location of the thenar muscles or eminence, and region 602 is the location of the hypo-thenar muscles eminence. The black dots 603 represent exemplary locations for placing the one or more stimulation pad 103. In order to provide ease-of-use for the patient, and to allow for different palm sizes, each stimulation pad 103 targets a larger section of a given muscle to be more effective for most hand sizes. By locating stimulation pads 103 near this unique location, when the muscles contract due to applied electrical stimulation, it forms a more natural and functional gripping action of the hand allowing the user to interact with and manipulate household items with ease. Region 604 shows an exemplary general region on a patient that device 100 targets for electrical stimulation. Region 605 shows another exemplary general region on a patient that device 100 targets for electrical stimulation.

Stimulation pads 103 may be described as having at least a first stimulation pad attached to a first location of garment 101. In some embodiments, device 100 comprises a first stimulation pad having an electrode and attached to a first location of the garment 101 and a second stimulation pad having an electrode and attached to a second location of garment 101, wherein, when garment 101 is positioned on the subject's hand, the first location of garment 101 is adjacent the thenar eminence of the subject's hand, and wherein the first stimulation pad is configured to deliver electrical stimulus to muscle tissue of the thenar eminence, and wherein, when garment 101 is positioned on the subject's hand, the second location of garment 101 is adjacent the hypothenar eminence of the subject's hand, and wherein the second stimulation pad is configured to deliver electrical stimulus to muscle tissue of the hypothenar eminence.

In some embodiments, device 100 comprises a third stimulation pad having an electrode and attached to a third location of garment 101, wherein a portion of garment 101 covers a region of the subject's arm, and the third location of garment 101 is adjacent the forearm of the subject's arm, and wherein the third stimulation pad is configured to deliver electrical stimulus to muscle tissue of the arm. In some embodiments, device 100 comprises a fourth stimulation pad having an electrode and attached to a fourth location of garment 101, wherein the fourth location of garment 101 is adjacent the forearm of the subject's arm, and wherein the fourth stimulation pad is configured to deliver electrical stimulus to muscle tissue of the arm. In some embodiments, third location is generally medial on the forearm, and fourth location is generally lateral on the forearm. In some embodiments, electrodes of the electrical stimulation pads positioned in the first and third locations of the flexible garment deliver electrical stimulus in a pair, and the electrodes in the second and fourth locations of the flexible garment deliver electrical stimulus in a pair.

In some embodiments, the first location and second location on the flexible garment are separated by a distance ranging between 1 cm and 10 cm. In some embodiments, the first location and third location, or the second and fourth location on the flexible garment are separated by a distance ranging between 4 cm and 50 cm. In some embodiments, the third location and fourth location on the flexible garment are separated by a distance ranging between 1 cm and 15 cm. In some embodiments, the first stimulation pad comprises a body having one or more curved surfaces configured to nest on a portion of the subject's hand. In some embodiments, the second electrical stimulation pad comprises a body having one or more curved surfaces configured to surround a portion of the subject's hand. In some embodiments, the third and fourth electrical stimulation pads each comprise a body having a convex surface.

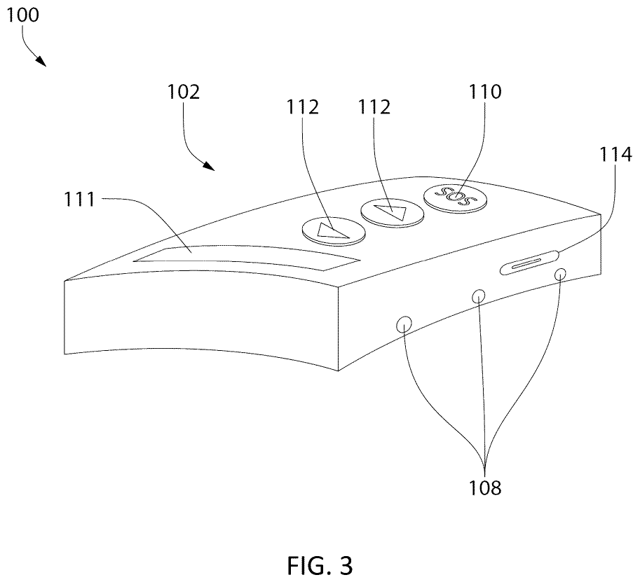

Referring now to FIG. 3, shown is an exemplary control unit 102 for device 100. In some embodiments, control unit 102 comprises a housing forming an interior volume, the housing having top and bottom surfaces, and one or more side surfaces. In some embodiments, control unit 102 comprises one or more string holes 108, or a plurality of holes, passing through the one or more side surfaces, forming a lumen in fluid connection with the interior volume of the housing. For example, in some embodiments, control unit 102 comprises a series of holes on a first side surface, and a series of holes on a second side surface diametrically opposed to the first series of holes on the first side surface. In some embodiments, plurality of strings 107 enter string holes 108, pass through the lumens of the holes, and enter the interior volume of the housing of control unit 102. In some embodiments, plurality of strings 107 connect to tightening mechanism 106, wherein the mechanism pulls the strings tight in order to tighten garment 101. In some embodiments, plurality of strings 107 connect to motor 106, wherein motor 106 is configured to rotate to pull the strings in order to tighten garment 101. In some embodiments, self-tightening mechanism 105 begins tightening when pressure sensor 115 detects a pressure below a threshold value, or within a pressure range. In some embodiments, self-tightening mechanism 105 ceases or stops tightening when pressure sensor 115 detects a pressure exceeding a threshold value, or within a pressure range. In some embodiments, self-tightening mechanism 105 is voice activated, and/or starts tightening when activated, or when a detected pressure is below a threshold value. In some embodiments, an optimal pressure range or thresholding for device 100 ranges between about 1 kg/cm² and about 2 kg/cm², or is at least 1 kg/cm², or is less than 5 kg/cm². In some embodiments, an optimal pressure value for device 100 is about 0.25 kg/cm², 0.5 kg/cm², 0.75 kg/cm², 1 kg/cm², 1.25 kg/cm², 1.5 kg/cm², 1.75 kg/cm², 2.00 kg/cm², 2.25 kg/cm², 2.5 kg/cm², 2.75 kg/cm², or about 3 kg/cm².

In some embodiments, control unit 102 comprises any of a display, a microphone, a speaker, a status indicator, an LED, a power button, a control button, an emergency stop button, a reset button, a power source, a battery, a charging port, a wireless charging antenna, a pressure sensor, a temperature sensor, and an IMU. For example, in some embodiments, control unit 102 comprises a microphone 109, emergency stop button 110, display 111, and one or more configuration buttons 112 positioned on the top surface of the housing. In some embodiments, control unit 102 comprises an opening in the top surface of the housing, wherein a mesh screen is positioned within the opening, and microphone 109 is positioned behind the mesh screen.

In some embodiments, device 100 and/or control unit 102 comprises one or more sensors. In some embodiments, one or more pressure sensors are positioned on or within device 100. For example, in some embodiments, control unit 102 comprises one or more pressure sensors positioned on the bottom surface of the housing. In some embodiments, garment 101 comprises one or more pressure sensors positioned on or within the garment. For example, in some embodiments, garment 101 comprises one or more pressure sensors 115 positioned on the inside surface of the garment, electrically connected to control unit 102, and configured to make contact with the skin of the patient and measure pressure (e.g., tissue interface pressure). In some embodiments, pressure sensor 115 is proximate, or distal to the at least one electrical stimulation pad 103, and in communication with the control unit. In some embodiments, pressure sensor 115 is positioned coaxial with stimulation pad 103, partially overlapping with stimulation pad 103, underneath stimulation pad 103, between stimulation pad 103 and garment 101, near stimulation pad 103, or any combination thereof. In some embodiments, the IMU detects movement of device 100 and powers on the device, a proximity sensor detects whether the wearable device is worn by the subject, the one or more pressure sensors read a current value, and adjust the tightening mechanism based on the read value.

In some embodiments, control unit 102 comprises one or more charging mechanisms. In some embodiments, control unit 102 comprises a wireless charging antenna. In some embodiments, control unit 102 comprises a charging mechanism 114 positioned in the one or more side surfaces of the housing.

Device 100 may be manufactured from or composed of various materials. For example, garment 101 may comprise any fabric known by one of ordinary level of skill in the art. Device 100 may use soft, flexible, biocompatible materials in any portion of the device. The electrical stimulation pads 103 (e.g., electrodes) may comprise any material or any configuration known in the art of electrodes and electrical stimulation. In some embodiments, garment 101 comprises cotton, polyester, nylon, wool, linen, or the like.

In some embodiments, control unit 102 comprises a computing system 700 including a processor and a non-transitory computer-readable medium with instructions stored thereon, which when executed by the processor perform steps comprising: receiving at least one voice command, actuating self-tightening mechanism 105 based on the at least one voice command, and applying electrical stimulation via the at least one electrical stimulation pad 103 based on the at least one voice command. In some embodiments, electrical stimulation pad 103 is configured to provide an electrical stimulation in the range 0-100 mA at 0-50 V. In some embodiments, electrical stimulation pad 103 is configured to provide an electrical stimulation at a 40 Hz frequency with 250 μS pulse width.

Aspects of the present disclosure relate to an electrical stimulation method, comprising providing any disclosed electrical stimulation device 100 of the present disclosure, receiving at least one voice command, affixing device 100 to a patient via self-tightening mechanism 105 based on the at least one voice command, and applying electrical stimulation via the at least one electrical stimulation pad 103 based on the at least one voice command.

In some embodiments, device 100 is controlled and/or configured with voice commands. For example, in some embodiments, voice commands from the subject are received to control unit 102 via microphone 109, wherein control unit 102 provides electrical stimulation to electrical stimulation pads 103, or provides electronic control to self-tightening mechanism 105.

In some embodiments, in order to activate or power on device 100, the user says the phrase “RIGHT GARMENT”, this can be modified to “LEFT GARMENT” for garments that are worn on the left side. After the activation phrase an LED light on control unit 102 and/or garment 101 lights up to indicate that the device is ready to receive commands from the user. In some embodiments, for example when the garment is configured as a glove, the action words to control the device are “GRASP”, “GRAB”, “HOLD”, “RELEASE”, “LEAVE”, “DROP”, “WEAR”, “REMOVE”. In some embodiments, control unit 102 stays awake for 20 seconds after activation and multiple commands can be given in that span. In some embodiments, after 20 seconds have passed, the user has to reactivate device 100 by saying “RIGHT GLOVE”. Upon receiving the command “GRASP”, “GRAB” or “HOLD”, control unit 102 gives a signal give electrical power to the electrical stimulation pads 103. In some embodiments, upon receiving the command “RELEASE”, “LEAVE” or “DROP”, control unit 102 stops the power to device 100 and/or electrical stimulation pads 103.

In some embodiments, using the command “WEAR” activates self-tightening mechanism 105. In some embodiments, motor 106 starts reeling in plurality of strings 107 thereby tightening garment 101. In some embodiments, motor 106 stops automatically once control unit 102 receives a signal from the one or more pressure sensor 115 indicating a threshold value has been reached. In some embodiments, the threshold value ranges between about 1-2 kg force on the one or more pressure sensor 115. In some embodiments, when the command “REMOVE” is used, motor 106 rotates in the opposite direction to open or loosen the garment.

Aspects of the present disclosure relate to a method of treating chronic weakness of the hand, comprising the steps of providing a wearable device to a subject comprising a flexible garment configured to fit around at least a portion of a subject, one or more electrical stimulation pads attached to the garment comprising at least a first electrical stimulation pad positioned over the thenar eminence, and a second electrical stimulation pad positioned over the hypothenar eminence, and providing electrical stimulation to the subject with the one or more electrical stimulation pads.

In some embodiments, the method further comprises the step of measuring interface pressure between the wearable device and the skin of the subject. In some embodiments, electrical stimulation is provided to the subject when the interface pressure is within a threshold range. In some embodiments, the method further comprises the step of providing electrical stimulation to a portion of the subject's flexor muscles with a third electrical stimulation pad positioned over an anterior portion of the subject's forearm.

In some embodiments, the method further comprises the step of providing electrical stimulation to a portion of the subject's flexor muscles with a fourth electrical stimulation pad positioned over an anterior portion of the subject's forearm, wherein the third electrical stimulation pad is positioned over a medial anterior portion of the forearm, and the fourth electrical stimulation pad is positioned over a lateral anterior portion of the forearm. In some embodiments, the first and third electrical stimulation pads are activated in a pair, and the second and fourth electrical stimulation pads are activated in a pair. In some embodiments, the method further comprises the step of tightening the wearable device with a voice-activated tightening mechanism attached to the flexible garment. In some embodiments, the method further comprises the step of activating the wearable device with a voice-activated control unit attached to the flexible garment.

### Experimental Examples

The disclosure is now described with reference to the following Examples. These Examples are provided for the purpose of illustration only and the disclosure should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present disclosure and practice the claimed methods. The following working examples therefore, specifically point out exemplary embodiments of the present disclosure, and are not to be construed as limiting in any way the remainder of the disclosure.

In some embodiments, the disclosed device is designed for patients with chronic hand weakness resulting from conditions such as stroke or spinal cord injury. One objective is to create ergonomic e-stim pads that exhibit exceptional durability by preventing corrosion. Additionally, these e-stim pads eliminate the necessity of adhering the pads to the patient's skin, thereby enhancing safety during usage.

In some embodiments, the device may operate through voice and touch commands, making it accessible to a broad spectrum of patients. In some embodiments, the device comprises one or more self-tightening mechanisms and boasts a compact design to enable independent usage.

A prototype of the device was made by using a wrist brace glove, an e-stim device, a micro-controller, voice recognition software, capacitive touch buttons, a motor, thin film pressure pads, and nylon string.

The design of the e-stim pads, and the placement of the pads make the pads more comfortable and induce a more natural gripping style as compared to other devices on the market. In order to make the e-stim pads, the anatomy of the hand was studied and the motion of the fingers and palm was observed to create a profile such that the pads put pressure on the thenar and hypo-thenar muscles of the hand. These pads were then 3D modelled in SolidWorks® and then 3D printed using PLA. A connecting wire was then laid on top of the pad and then covered with copper tape, acting as the electrode of the e-stim pad. The other end of the wire was connected to the e-stim device. The e-stim device was a Neuro Muscular Electric Stimulation (NMES) device made by Balego®. The device was set to 40 Hz frequency with 250 us pulse width. The e-stim device has an adjustable range for current from 0-100 mA at 0-50 V which is adjustable with a 5 pin potentiometer. For the disclosed device, the e-stim device was modified so that it can be controlled by an Arduino microcontroller and a capacitive touch potentiometer to control the current from the device to suit the users need. The e-stim device was set to continuous mode and the Arduino controls the power going to the device in order to induce stimulation to the muscle when the user needs it. Two pressure sensors were placed underneath the pads and then the pads were then stuck in the position below the correct muscles in the glove.

The device is controlled by an Arduino Nano RP2040 Connect microcontroller that uses the Arduino voice recognition library to recognize voice commands. To activate the device, the user says the phrase “RIGHT GLOVE”, this can be modified to “LEFT GLOVE” for gloves that are worn on the left hand. After the activation phrase an LED lights up to indicate that the device is ready to receive commands from the user. The action words to control the device are “GRASP”, “GRAB”, “HOLD”, “RELEASE”, “LEAVE”, “DROP”, “WEAR”, “REMOVE”. The Arduino stays awake for 20 seconds after activation and multiple commands can be given in that span. After 20 seconds have passed, the used has to reactivate the device by saying “RIGHT GLOVE”. Upon receiving the command “GRASP”, “GRAB” or “HOLD”, the Arduino gives a signal to a relay to give power to the e-stim device. Upon receiving the command “RELEASE”, “LEAVE” or “DROP”, the Arduino stops the power to e-stim device.

Using the command “WEAR” starts the self-tightening mechanism. A continuous rotation servo motor starts reeling in the nylon strings thereby tightening the glove. The motor stops automatically once the Arduino receives a signal from the pressure sensors indicating about 1-2 kg force on both pads. When the command “REMOVE” is used, the motor rotates in the opposite direction to open the glove.

The wireless charging pad is mounted on the underside of the glove and the wires from it run directly to the battery thus charging it when kept on a wireless charger.

An emergency stop button is given to stop the device from working completely in case the user is having any trouble. Once the emergency switch is triggered, to reset the device, the reset button on the Arduino can be pressed through a reset hole using a paper clip and that will reset and restart the device.

A case is designed to fit all the electronics and with a mesh opening for the microphone to receive voice commands. The components are all fit inside with the cables from the pads and wireless charger going into it through dedicated holes in the case. The self-tightening string also come into the case and are connected to the motor. Once the case is assembled, it is stuck on top of the glove.

In some embodiments, the e-stim pad design uses an ergonomic design based on the natural curvature of the hand. In some embodiments, the e-stim pad uses copper plating as the electrodes which are pressed onto the skin with some pressure to induce current through NMES.

Existing technologies in place many pads along the arm to trigger a tenodesis grasp which is not the way humans grasp things naturally. By placing the pads on the Thenar and Hypo-thenar muscles in the hand, the grip induced is much more natural and functional. The same concept of making pads with copper plating can be extended to incorporate other pad placements as well to suit a patient's specific needs.

Another example is provided herein. In some embodiments, the device is an electric stimulation device used to stimulate the muscles in the hands of patients with chronic weakness due to stroke or spinal cord injury. The prototype was built using readily available technologies and components such as a wrist brace glove, NMES e-stim device, Arduino micro-controller, touch screen module, thin-film pressure sensor, ratcheting mechanism, nylon/elastic laces, and various CAD, manufacturing, and programming software. The innovation in this device lies in the design of the e-stim pads and their placement to make them more comfortable and induce a more natural gripping style compared to other devices on the market.

To design the e-stim pads, the anatomy of the hand was thoroughly studied, and the motion of the fingers and palm was observed to create a profile that puts pressure on the thenar and hypo-thenar muscles of the hand. These pads are then 3D modeled in SolidWorks®. The pad design consists of two parts that clip into each other. The top piece is made from stainless steel, and the base of the pad is made from PLA plastic. Spiral grooves with a 1 mm diameter are incorporated on the bottom of the top piece and the top of the base to facilitate wire routing. The base also features a swept hole to conceal the wire.

The top piece of the pads was manufactured using a selective laser sintering (SLS) metal 3D printer with Stainless steel (SS-306). After printing, the pads were finished by grinding the surface that comes in contact with the user's skin to achieve a smooth, shiny finish. The base of the pad was manufactured using a plastic extrusion 3D printer with PLA plastic filament. Copper tape was embedded into the grooves on the bottom of the top piece of the pad to allow for soldering wires. A section of thin electrical wire was stripped of its sheath and routed through the holes in the base. The wire was carefully soldered into the groove, and excess solder was ground to create a flush interface between the top piece and the base.

A third pad was designed in the shape resembling a button and was placed along the forearm. This pad also features spiral grooves for wire routing, and the wire was soldered in the same way as in the previous case. The wire from one of the previous pads was split, and the two pads were connected in series. The other end of the wire was connected to the e-stim device, which was a Neuro Muscular Electric Stimulation (NMES) device made by Balego®. It was set to a 40 Hz frequency (adjustable range: 2-150 Hz) with a 250 μS pulse width (adjustable range: 50-300 μS) for the safety of the patient, but these values can be changed by the therapist remotely based on the patient's profile. These settings are only accessible to the therapist due to safety concerns related to improper use of electricity through the body. The current has an adjustable range of 0-100 mA at 0-50 V, which the user can change locally on the touch screen. This setting is user-accessible.

The e-stim device was modified so that it can be controlled by an Arduino micro-controller and a touch screen. The e-stim device was set to continuous mode, and the Arduino controls the power going to the device to induce muscle stimulation when the user needs it.

The device is controlled by an Arduino Nano RP2040 Connect microcontroller, which uses the Arduino voice recognition library to recognize voice commands. To activate the device, the user says the phrase “RIGHT GLOVE,” which can be modified to “LEFT GLOVE” for gloves worn on the left hand. After the activation phrase, an LED lights up to indicate that the device is ready to receive commands from the user. The action words used to control the device are “GRASP,” “GRAB,” “HOLD,” “RELEASE,” “LEAVE,” “DROP,” “WEAR,” and “REMOVE.” The Arduino stays awake for 20 seconds after activation, and multiple commands can be given within that span. After 20 seconds have passed, the user must reactivate the device by saying “RIGHT GLOVE.” Upon receiving the command “GRASP,” “GRAB,” or “HOLD,” the Arduino sends a signal to a relay to provide power to the e-stim device. When the command “RELEASE,” “LEAVE,” or “DROP” is received, the Arduino stops the power to the e-stim device.

Using the command “WEAR” initiates the self-tightening mechanism. A continuous rotation servo motor starts reeling in the nylon strings with a ratchet mechanism, tightening the glove. Two thin-film pressure sensors are placed under the pads and halt the self-tightening mechanism when they register a pressure of 1-2 kg/cm² on both pads. The command “REMOVE” causes the motor to rotate in the opposite direction, opening the glove.

The device can also be controlled using Electromyography signals. The same pads used to deliver e-stim are also used as EMG sensors. The Arduino controller employs a machine learning algorithm to detect these signals and classify them into categories: GRAB, RELEASE, WEAR, and REMOVE.

A wireless charging pad is mounted on the underside of the glove, and the wires from it run directly to the battery, charging it when placed on a wireless charger. An emergency stop button is provided on the touch screen to completely halt the device's operation in case the user encounters any trouble.

A case is designed to house all the electronics with a mesh opening for the microphone to receive voice commands. The components fit inside, and the cables from the pads and wireless charger enter through dedicated holes in the case. The self-tightening strings are also connected to the motor inside the case. Once assembled, the case is affixed to the glove and is manufactured using a plastic extrusion 3D printer with PLA plastic filament.

The placement of the pads is crucial, with one pad permanently affixed under the thenar muscle of the palm and another under the hypo-thenar muscle of the palm, both on the glove. These pads incorporate thin-film pressure sensors. The third pad is stitched along the forearm on the glove. The pad design accommodates hands of all shapes and sizes and serves as EMG sensors to detect signals from the hand.

The device was tested by the inventors on themselves and verified the functionality of the device. Verified the correct muscle groups were getting activated and the hand was making a grasping motion. Checked the strength of grip by lifting common household items like coffee mug, water bottle, books, etc.

The current e-stim pads using the TENS method require to be stuck to the skin and pose a risk of injury if the pads are dry and not conducting. The disclosed device, being compact, portable, and easy-to-use, solves these problems faced by users. The e-stim pads are ergonomic, developed using SLS metal 3D printing procedures, and made of stainless steel SS-306. They utilize the natural curvature of the hand with stainless steel to work as electrodes, inducing current through the NEMS method, giving more freedom to the users, and providing a “natural” grip. Another aspect of the device is the use of IoT, AWS, and sensors to connect the users with the therapists in real time. Additional touch screen and voice activation offer a wide range of operating principles.

Self-tightening was achieved using a combination of motors and a rachet mechanism. In order to make it easier for the user and allow for different palm sizes, the unique pad design targets a larger section of the muscle to be more effective for most hand sizes. Two pads in the device are located in approximately the middle of the thenar and hypothenar muscles of the palm, respectively, whereas the third pad is located along the forearm to assist in achieving the primary objective. This specific placement ensures a more natural and functional grip, allowing the user to manipulate any item with ease.

This device uses e-stim pads that do not need to be changed or stuck to the skin. Along with this crucial pad design, the voice and touch activation, larger area of contact for the pads, and self-tightening mechanism make this device unique from the current products. Additionally, functional aspects like self-charging and easy donning and doffing make this device more suitable for independent use.

This device makes the life of patients much more comfortable and gives them more independence and control over their lives. This is very important as improving quality of life of patients with chronic ailments is essential to their families and themselves. Using this disclosure many people will be able to get a device that is affordable, easy to use and maintain, comfortable and can make the users more independent.

### Computing Environment

In some aspects of the present disclosure, software executing the instructions provided herein may be stored on a non-transitory computer-readable medium, wherein the software performs some or all of the steps of the present disclosure when executed on a processor.

Aspects of the disclosure relate to algorithms executed in computer software. Though certain embodiments may be described as written in particular programming languages, or executed on particular operating systems or computing platforms, it is understood that the system and method of the present disclosure is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PHP, Perl, Ruby, or Visual Basic. It is further understood that elements of the present disclosure may be executed on any acceptable computing platform, including but not limited to a server, a cloud instance, a workstation, a thin client, a mobile device, an embedded microcontroller, a television, or any other suitable computing device known in the art.

Parts of this disclosure are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present disclosure, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this disclosure are described as communicating over a variety of wireless or wired computer networks. For the purposes of this disclosure, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the disclosure may be implemented over a Virtual Private Network (VPN).

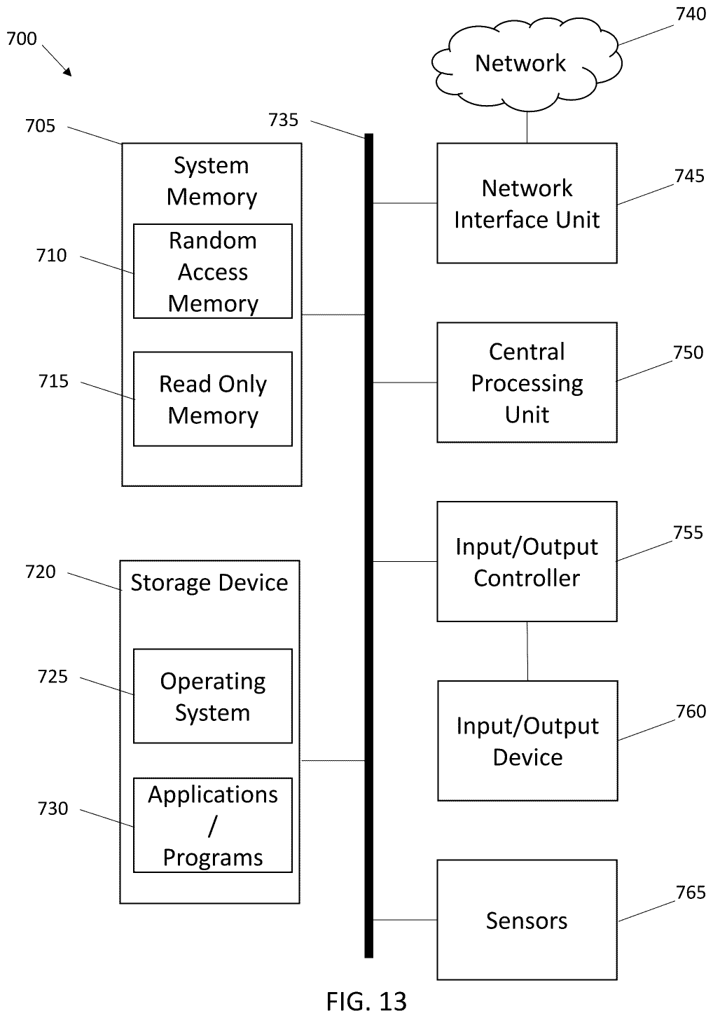

FIG. 13 and the following discussion are intended to provide a brief, general description of a suitable computing environment in which the disclosure may be implemented. While the disclosure is described above in the general context of program modules that execute in conjunction with an application program that runs on an operating system on a computer, those skilled in the art will recognize that the disclosure may also be implemented in combination with other program modules.

Generally, program modules include routines, programs, components, data structures, and other types of structures that perform particular tasks or implement particular abstract data types. Moreover, those skilled in the art will appreciate that the disclosure may be practiced with other computer system configurations, including hand-held devices, multiprocessor systems, microprocessor-based or programmable consumer electronics, minicomputers, mainframe computers, and the like. The disclosure may also be practiced in distributed computing environments where tasks are performed by remote processing devices that are linked through a communications network. In a distributed computing environment, program modules may be located in both local and remote memory storage devices.

FIG. 13 depicts an illustrative computer architecture for a computer 700 for practicing the various embodiments of the disclosure. The computer architecture shown in FIG. 13 illustrates a conventional personal computer, including a central processing unit 750 (“CPU”), a system memory 705, including a random-access memory 710 (“RAM”) and a read-only memory (“ROM”) 715, and a system bus 735 that couples the system memory 705 to the CPU 750. A basic input/output system containing the basic routines that help to transfer information between elements within the computer, such as during startup, is stored in the ROM 715. The computer 700 further includes a storage device 720 for storing an operating system 725, application/program 730, and data.

The storage device 720 is connected to the CPU 750 through a storage controller (not shown) connected to the bus 735. The storage device 720 and its associated computer-readable media, provide non-volatile storage for the computer 700. Although the description of computer-readable media contained herein refers to a storage device, such as a hard disk or CD-ROM drive, it should be appreciated by those skilled in the art that computer-readable media can be any available media that can be accessed by the computer 700.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the disclosure, the computer 700 may operate in a networked environment using logical connections to remote computers through a network 740, such as TCP/IP network such as the Internet or an intranet. The computer 700 may connect to the network 740 through a network interface unit 745 connected to the bus 735. It should be appreciated that the network interface unit 745 may also be utilized to connect to other types of networks and remote computer systems.

The computer 700 may also include an input/output controller 755 for receiving and processing input from a number of input/output devices 760, including a keyboard, a mouse, a touchscreen, a camera, a microphone, a controller, a joystick, or other type of input device. Similarly, the input/output controller 755 may provide output to a display screen, a printer, a speaker, or other type of output device. The computer 700 can connect to the input/output device 760 via a wired connection including, but not limited to, fiber optic, ethernet, or copper wire or wireless means including, but not limited to, Bluetooth, Near-Field Communication (NFC), infrared, or other suitable wired or wireless connections.

As mentioned briefly above, a number of program modules and data files may be stored in the storage device 720 and RAM 710 of the computer 700, including an operating system 725 suitable for controlling the operation of a networked computer. The storage device 720 and RAM 710 may also store one or more applications/programs 730. In particular, the storage device 720 and RAM 710 may store an application/program 730 for providing a variety of functionalities to a user. For instance, the application/program 730 may comprise many types of programs such as a word processing application, a spreadsheet application, a desktop publishing application, a database application, a gaming application, internet browsing application, electronic mail application, messaging application, and the like. According to an embodiment of the present disclosure, the application/program 730 comprises a multiple functionality software application for providing word processing functionality, slide presentation functionality, spreadsheet functionality, database functionality and the like.

The computer 700 in some embodiments can include a variety of sensors 765 for monitoring the environment surrounding and the environment internal to the computer 700. These sensors 765 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure.

What is claimed is:

1. A wearable device, comprising:
a flexible garment configured to fit around at least a portion of a subject's hand;
a first stimulation pad having an electrode and attached to a first location of the flexible garment; and
a second stimulation pad having an electrode and attached to a second location of the flexible garment;
wherein, when the flexible garment is positioned on the subject's hand, the first location of the flexible garment is adjacent the thenar eminence of the subject's hand, and wherein the first stimulation pad is configured to deliver electrical stimulus to muscle tissue of the thenar eminence, and
wherein, when the flexible garment is positioned on the subject's hand, the second location of the flexible garment is adjacent the hypothenar eminence of the subject's hand, and wherein the second stimulation pad is configured to deliver electrical stimulus to muscle tissue of the hypothenar eminence.

2. The device of claim 1, further comprising a tightening unit, the tightening unit comprising a control unit and a plurality of tightening lines, wherein the plurality of tightening lines are connected to the control unit and the flexible garment, and wherein the control unit is configured to adjust the tension on the plurality of tightening lines.

3. The device of claim 2, further comprising one or more pressure sensors attached to the flexible garment and configured to measure pressure on the skin of the subject, wherein the one or more sensors are communicatively connected to the control unit.

4. The device of claim 3, wherein the control unit is voice-activated for adjusting the tension on the plurality of tightening lines and activating the electrodes of the stimulation pads.

5. The device of claim 4, wherein the control unit adjusts the tension on the plurality of tightening lines based on one or more pressure signals received from the one or more pressure sensors.

6. The device of claim 5, wherein the tightening unit comprises a housing having a plurality of holes, wherein the plurality of tightening lines extend through the plurality of holes to connect to the control unit positioned inside the housing.

7. The device of claim 6, wherein the control unit comprises at least one motor connected to the plurality of tightening lines within the housing, wherein the at least one motor is configured to rotate to adjust the tension on the plurality of tightening lines.

8. The device of claim 7, wherein the tightening unit and the plurality of tightening lines are removably connected to the flexible garment.

9. The device of claim 1, wherein the first stimulation pad comprises a body having one or more curved surfaces configured to nest on a portion of the subject's hand.

10. The device of claim 9, wherein the second stimulation pad comprises a body having one or more curved surfaces configured to surround a portion of the subject's hand.

11. The device of claim 10, wherein the flexible garment comprises a cut-out region for the thumb of the subject's hand.

12. The device of claim 1, further comprising a third stimulation pad having an electrode and attached to a third location of the flexible garment, wherein a portion of the flexible garment covers a region of the subject's arm, and the third location of the flexible garment is adjacent the forearm of the subject's arm, and wherein the third stimulation pad is configured to deliver electrical stimulus to muscle tissue of the arm.

13. The device of claim 12, further comprising a fourth stimulation pad having an electrode and attached to a fourth location of the flexible garment, wherein the fourth location of the flexible garment is adjacent the forearm of the subject's arm, and wherein the fourth stimulation pad is configured to deliver electrical stimulus to muscle tissue of the arm.

14. The device of claim 13, wherein the third location of the flexible garment is adjacent the medial forearm of the subject's arm, and the fourth location of the flexible garment is adjacent the lateral forearm of the subject's arm.

15. The device of claim 13, wherein the electrodes of the stimulation pads positioned in the first and third locations of the flexible garment deliver electrical stimulus in a pair, and the electrodes in the second and fourth locations of the flexible garment deliver electrical stimulus in a pair.

16. The device of claim 13, wherein the third and fourth stimulation pads each comprise a body having a convex surface.

17. The device of claim 13, wherein the first location and second location on the flexible garment are separated by a distance ranging between 1 cm and 10 cm.

18. The device of claim 17, wherein the first location and third location on the flexible garment are separated by a distance ranging between 4 cm and 50 cm, and the third location and fourth location on the flexible garment are separated by a distance ranging between 1 cm and 15 cm.

19. The device of claim 18, wherein the electrodes of the stimulation pads are configured to deliver an electrical stimulus in the range 0-100 mA at 0-50 V.

20. The device of claim 19, wherein the electrodes of the stimulation pads are configured to deliver an electrical stimulus at a 40 Hz frequency with 250 μS pulse width.
